(19) United States
(12) Patent Application Publication
(10) Pub. No.: US 2009/0143707 A1
(43) Pub. Date: Jun. 4, 2009

(54) Knee heat/massage device

(76) Inventor: James R. Strahl, Indianapolis, IN (US)

Correspondence Address: JAMES R STRAHL, 1704 MACE DRIVE, INDIANAPOLIS, IN, 46229, US

(21) Appl. No.: 11/987,600

(22) Filed: Dec. 3, 2007

(51) Int. Cl. A61H 7/00; A61F 5/00; A61H 1/00

(52) U.S. Cl. 601/134; 601/18; 602/2

(57) ABSTRACT

A massage pad with heat that wraps to the knee joint providing massage therapy and heat. The knee pad is designed to be wrapped securely around the knee; this holds the massaging and heating elements against the knee joint while the pad is designed to allow bending of the knee for individual use. The pad is controlled with a controller for selecting the degree of heat and one or more massage actuators to provide a variety of massaging to the knee.

### FIELD OF THE INVENTION

The present invention relates to a heat/massage device comprising of a heat element and massaging elements which can be secured around the knee providing heat and massage therapy.

### BACKGROUND OF THE INVENTION

Heating pads are used for treating muscle and joint pain. Massage therapy can further sooth and relax individuals. Massage pads having a number of individual actuators are well known in the art. Such cushions may include a series of raised portions or nodes under which lies an actuator such as a vibrating motor.

Vibrating massage pads of the prior art are typically in the form of a seat cushion having a lower seating position and a back rest position. A variety of actuators are placed to accommodate the users. In addition a different zones on the cushion may incorporate one or more actuators and be individually energized. The controller may also allow a user to vary the intensity and sequence of vibration of various actuators.

However, no prior art device is designed to be used specifically around the knee joint. Whereas some prior art allows for wrapping, they do not provide the flexibility while staying secured to the knee joint.

### SUMMARY OF THE INVENTION

It is an advantage for a heat/massage device to have a shape that can be custom-fit to the individual users knee joint. A releasable complementary fastening system is used to secure the device around the users knee joint.

It is an advantage for the heat to be separately controlled allowing the user to turn on or off the heat as desired by the user.

The massage actuators are placed along each side of the knee joint allowing the massage to occur with the knee in a number of positions or cycles selected by the user.

An objective of the present invention is to allow the individual to control heat and massaging by activating actuators and controlling their cycle.

### BRIEF DESCRIPTION OF THE DRAWINGS

FIG. 1 is a perspective view of a leg with the knee heat/massage device wrapped around the knee, which is plugged into a control unit.

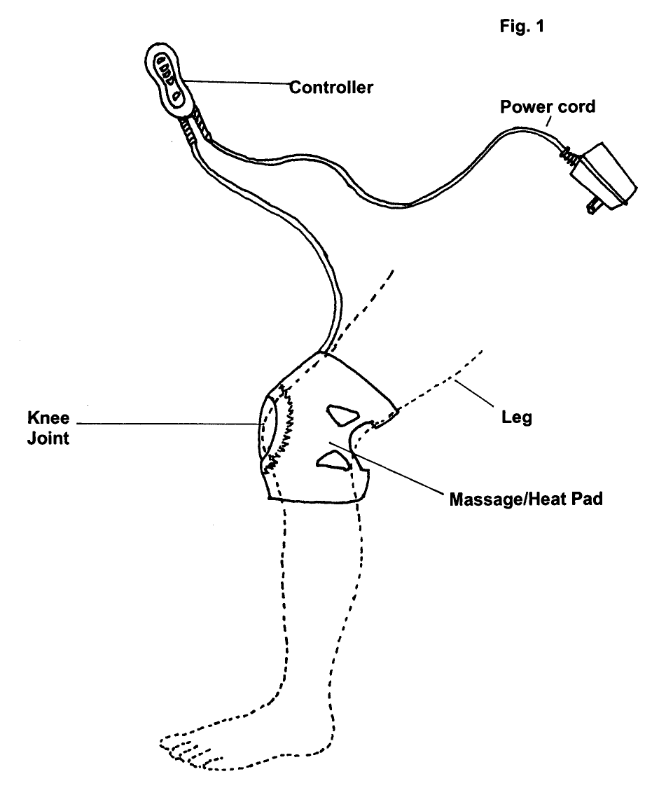

FIG. 2 is a top perspective of the knee heat/massage device with the attached control unit.

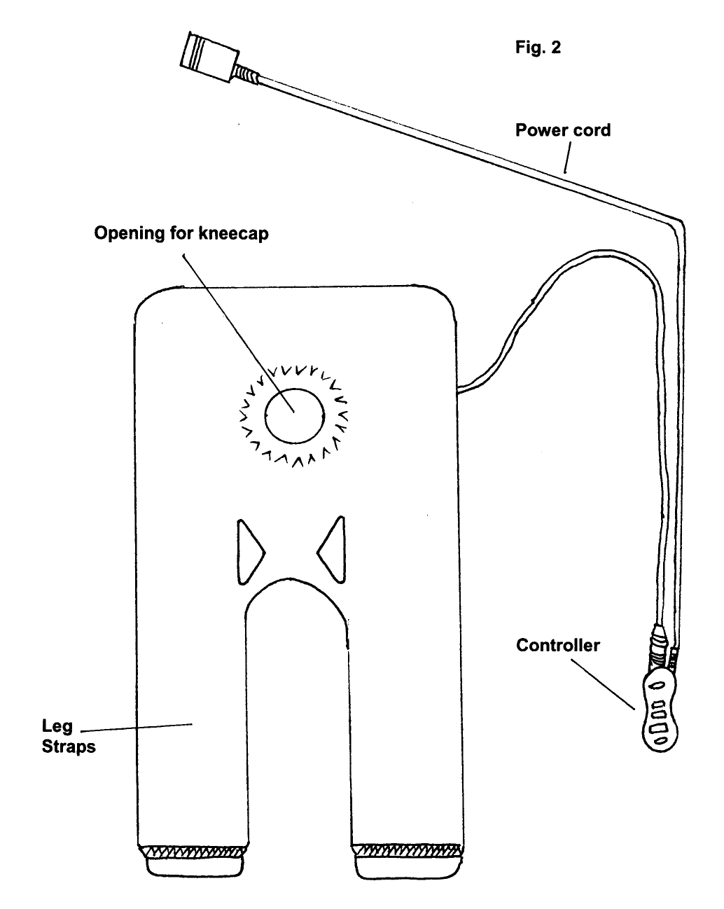

FIG. 3 is a bottom perspective of the knee heat/massage device showing the location of the massage actuators and heating element attached to the control unit.

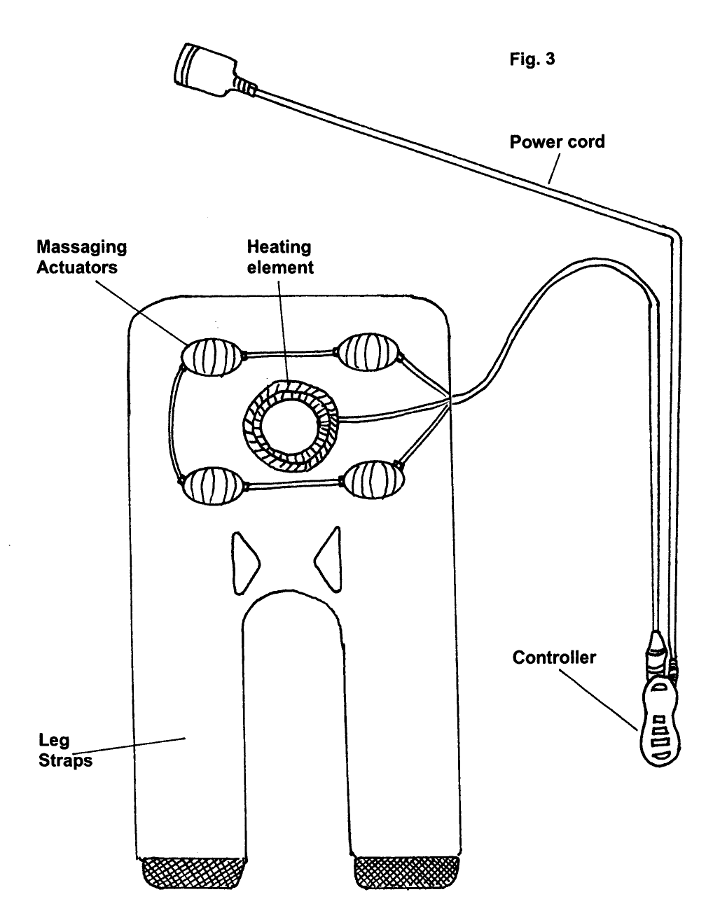

1. A heat/massage knee device for physical therapy comprising: a control unit connected to a power source, the control unit having switches with variable positions to; control the temperature of the heat, degree of vibration for each massage actuator and the cycle of the massaging actuators.

2. The knee heat/massage pad of claim 1 wherein the first flexible heat/massage device designed for the knee joint.

3. The knee heat/massage device is made of resilient material designed with integrated velcro at the end of the straps to allow individuals to strap around the knee and attach to the other side of the knee. The straps at the bottom and top of the pad allow for the knee to be bent while the heat and massage pad is held secure against the knee.

4. The control unit consists of several switches that control the power, heat, and massaging actuators.

5. From claim 1, massaging actuators are vibrating motors positioned laterally along each side of the knee device.

6. From claim 1, the temperature around the knee can be heated using an integrated heating pad around the knee device.
